(19) United States
(12) Patent Application Publication
(10) Pub. No.: US 2005/0196356 A1
(43) Pub. Date: Sep. 8, 2005

(54) Bleaching preparation and applicators for the teeth

(76) Inventors: Hans-Theo Leinen, Erkrath (DE); Markus Hunold, Duisburg (DE); Nadia Ben-Abderrahmane, Ecully (FR)

Correspondence Address: HENKEL CORPORATION, THE TRIAD, SUITE 200, 2200 RENAISSANCE BLVD., GULPH MILLS, PA, 19406, US

(21) Appl. No.: 10/986,731

(22) Filed: Nov. 12, 2004

(30) Foreign Application Priority Data
Mar. 8, 2004 (DE) 10 2004 011 557.5

(51) Int. Cl. A61K 7/20

(52) U.S. Cl. 424/53

(57) ABSTRACT

A preparation for bleaching teeth, comprising 0.3 to 30% by weight of a bleaching component selected from hydrogen peroxide or carbamide peroxide, 4 to 80% by weight of water, 0.05 to 1.00% by weight of a complexing agent selected from the group of the biphosphonates, and 0.1 to 3.0% by weight of a thickener, the preparation exhibiting a pH value in the range from 4 to 7 and a viscosity in the range from 1000 to 120 000 mPa·s (measured with a Brookfield RVF; up to 40 000 mPa·s spindle 4/4 rpm; from 40 000 mPa·s spindle TE/4 rpm; Helipath), the preparation being used in particular in an applicator which exhibits an applicator head which can be impregnated with the preparation to act upon the surfaces of the teeth of the user.

### CROSS REFERENCE TO RELATED APPLICATION

This application claims priority of DE 102004011557.5, filed Mar. 8, 2004, which is incorporated herein by reference in its entirety.

### BACKGROUND OF THE INVENTION

The present invention relates to a preparation for bleaching the teeth and to the use thereof in specific applicators.

The use of peroxides as component in oral and dental care preparations has already been known for a very long time, first since peroxide compounds exhibit an antimicrobial action against many harmful microorganisms of the oral cavity and, in this way, contribute to the treatment of gingivitis and periodontitis and to the control of dental plaque. Secondly, peroxides, because of their bleaching action, bring about a brightening of darkly discolored teeth and accordingly contribute substantially to the cleaning success of oral and dental care preparations.

One disadvantage of peroxide-comprising preparations is the lack of stability of peroxides in aqueous solution, which can result in a loss of peroxide active oxygen in the course of fairly lengthy storage, in particular at fairly high ambient temperatures. This problem is particularly serious if oxidizable organic components or components which give rise to a neutral or weakly basic pH value or else require activation of the peroxide oxygen are present in the preparation.

An additional known problem is that peroxides are rather unspecific in their oxidizing action and accordingly can result, at fairly high dosing, such as would be desirable for the bleaching of discolored teeth, in burning of the oral mucous membrane. For this reason, upper concentration limits for the use of, e.g. hydrogen peroxide, in oral hygiene preparations have been specified by law in many countries.

Attempts have accordingly not been lacking to stabilize peroxide-comprising oral and dental care preparations against peroxide decomposition in order to obtain a satisfactory action even at all concentrations of use over a fairly lengthy time.

In addition, attempts have been made for a long time to find suitable application forms for dental bleaching agents which make it possible to apply the dental bleaching agent to the teeth with the greatest possible accuracy in order thus not to damage the gum or thus to damage the gum as little as possible. In this connection, it is advantageous, depending on the application form, to make available stable formulations over a viscosity range which is as great as possible. In this way, each user can individually select the application forms suitable for him and preferred by him which comprise either formulations with high viscosity or those with low viscosity.

The critical pH value, at which the mineral losses (demineralization) of the dental enamel exceed the capability of reincorporating minerals (remineralization), lies between 5.4-5.7 for the dental enamel. Since a fairly lengthy close contact of applied bleaching agent and dental enamel occurs in the dental bleaching, it is accordingly furthermore desirable to allow the pH value of the bleaching agent to lie as far as possible in the neutral region in order to minimize as far as possible the process of demineralization.

A preparation for the bleaching of the teeth has now surprisingly been found which can be formulated in a stable way over a broad viscosity range and is accordingly suitable for use in a multitude of different application forms. The abovementioned requirements are accordingly to a large extent fulfilled.

The present invention accordingly relates to a preparation for bleaching teeth, comprising
0.3 to 30% by weight of a bleaching component selected from hydrogen peroxide or carbamide peroxide,
4 to 80% by weight of water,
0.05 to 1.00% by weight of a complexing agent selected from the group of the biphosphonates, and
0.1 to 3.0% by weight of a thickener,
   which exhibits a pH value in the range from 4 to 7 and a viscosity in the range from 1000 to 120 000 mPa·s (measured with a Brookfield RVF; up to 40 000 mPa·s spindle 4/4 rpm; from 40 000 mPa·s spindle TE/4 rpm; Helipath).

Those preparations are preferred according to the invention in which the content of the bleaching component is 3 to 18% by weight.

Furthermore, preferred preparations according to the invention exhibit a water content of 5 to 65% by weight.

The alkali metal salts of azacycloheptane-2,2-diphosphonic acid or of 1-hydroxyethane-1,1-diphosphonic acid act as suitable biphosphonates according to the invention.

Preference is given, within the meaning of the invention, to those preparations for bleaching the teeth which exhibit a pH value in the range from 6 to 7.

In a preferred embodiment of the invention, the preparations for bleaching the teeth exhibit a water content of 45 to 65% by weight and a viscosity [mPa·s] in the range from 8000 to 45 000 (measured with a Brookfield RVF; spindle 4/4 rpm).

In an additional preferred embodiment of the invention, the preparations for bleaching the teeth exhibit a water content of 5 to 25% and a viscosity [mPa·s] in the range from 8000 to 45 000 (measured with a Brookfield RVF; spindle 4/4 rpm).

The bleaching component is selected, according to the invention, from hydrogen peroxide and/or carbamide peroxide and is present in the dental bleaching preparations at a maximum content of 30% by weight. Examples of suitable bleaching components are the 30% H₂O₂ solution (H₂O₂ content of at least 35% by weight; active oxygen content of at least 16.5% by weight) sold under the trade name Perhydrol® by Merck KGaA or carbamide peroxide, for example sold by Degussa.

The complexing agent is selected, according to the invention, from the group of the biphosphonates, in particular from alkali metal salts of azacycloheptane-2,2-diphosphonic acid or of 1-hydroxyethane-1,1-diphosphonic acid.

Particular preference is given, within the meaning of the invention, to the disodium salt of azacycloheptane-2,2-diphosphonic acid.

The thickener is usually polymers from the group of the crosslinked polyacrylic acids (carbopols)

Suitable carbopols according to the invention are sold by Noveon under the trade names Carbopol 980 NF, Carbopol 971P NF or Carbopol 974.

In a particularly preferred embodiment of the invention, the thickener is selected from water-dispersible polymers of polyacrylic acid crosslinked with allylpentaerythritol (Carbopol 974 NFP).

In a preferred embodiment, the preparations according to the invention furthermore comprise at least one additional binder or thickener. These act by regulating the consistency and furthermore prevent the separation of the liquid and solid constituents.

The amounts thereof used in the compositions according to the invention are 0.1-5% by weight, preferably 0.1-3% by weight and in particular 0.5-2% by weight.

Use is made according to the invention, for example, of natural and/or synthetic water-soluble polymers, such as alginates, carrageenans, agar, guar gum, gum arabic, succinoglycan gum, guar flour, carob bean flour, gum tragacanth, karaya gum, xanthan, pectins, cellulose and its ionogenic and non-ionogenic derivatives, such as, for example, carboxymethylcellulose, hydroxyethylcellulose or methylhydroxypropylcellulose, hydrophobically modified celluloses, starch and starch ethers.

Polyvinyl alcohol, polyvinylpyrrolidone and polymolecular polyethylene glycols (in particular those with molecular weights of 10²-10⁶ D) are also suitable as binder or thickener. Layered silicates and finely divided silicas (aerogel silicas and pyrogenic silicas) can likewise fulfill this function.

Water-insoluble underivatized celluloses, for example sold by J. Rettenmaier & Sohne under the description Arbocel® and Vitacel®, are preferably suitable as binder or thickener. Within the meaning of the invention, the term “water-insoluble” is understood to mean a solubility of less than 1% by weight in water at 20° C., i.e. that less than 1% by weight of the cellulose is dissolved in 100 g of a saturated solution at 20° C.

Arbocel® CGP 5000, a high-viscosity paste formed from cellulose powder with thixotropic properties, is a particularly effective thickener which even at a low concentration of use has strong consistency-conferring properties, is inert with regard to ionic constituents and can be readily combined with additional thickeners.

Moisturizers are generally used in dental cosmetics for protecting the products from drying out, for regulating the consistency of the products and for the stability toward cold of the products. However, they can also furthermore be used for mediating the suspension and for influencing the taste or sheen.

Use is usually made, as moisturizers, of toxicologically harmless polyols, such as, for example, sorbitol, xylitol, glycerol, mannitol, 1,2-propylene glycol or mixtures thereof, but polyethylene glycols with molecular weights of 400-2000 can also be used as moisturizing components in oral and dental treatment preparations.

The combination of several moisturizing components is preferred, the combination of glycerol, 1,2-propylene glycol and/or polyethylene glycol being able to be regarded as particularly preferred.

Depending on the type of product, the moisturizer or the mixture of moisturizers is present in the combined composition in an amount of 10-85% by weight, preferably 20-70% by weight and in particular 30-50% by weight.

It can be advantageous according to the invention, in particular for dissolving the mostly water-insoluble aromatic oils, to use a non-ionogenic solubility promoter from the group of the surface-active compounds. Particularly suitable for this purpose are, e.g., oxyethylenated fatty acid glycerides, oxyethylenated fatty acid sorbitan partial esters or fatty acid partial esters of glycerol or sorbitan ethoxylates. Solubility promoters from the group of the oxyethylenated fatty acid glycerides include above all addition products of 20 to 60 mol of ethylene oxide with mono- and diglycerides of linear fatty acids having 12 to 18 carbon atoms or with triglycerides of hydroxy fatty acids, such as hydroxystearic acid or ricinoleic acid. Additional suitable solubility promoters are oxyethylenated fatty acid sorbitan partial esters which are preferably addition products of 20 to 60 mol of ethylene oxide with sorbitan monoesters and sorbitan diesters of fatty acids having 12 to 18 carbon atoms. Likewise suitable solubility promoters are fatty acid partial esters of glycerol or sorbitan ethoxylates which are preferably mono- or diesters of C₁₂-C₁₈ fatty acids and addition products of 20 to 60 mol of ethylene oxide with 1 mol of glycerol or with 1 mol of sorbitol.

The dental bleaching preparations according to the invention preferably comprise, as solubility promoter for optionally present aromatic oils, addition products of 20 to 60 mol of ethylene oxide with hydrogenated or nonhydrogenated castor oil (i.e., with hydroxystearic acid or ricinoleic acid triglyceride), with glycerol mono- and/or with sorbitan mono- and/or distearate.

An additional preferred embodiment of the dental bleaching preparation according to the invention is one in which the preparation comprises, as additional active substances, active substances for combating tooth decay, antimicrobial active substances, scale inhibitors, remineralization active substances, flavoring agents, sweeteners or any combination of these substances.

Active substances for combating tooth decay

Fluorine compounds, preferably from the group of the fluorides or monofluorophosphates, in an amount of 0.1-0.5% by weight of fluorine, are suitable above all for combating and preventing tooth decay. Suitable fluorine compounds are, e.g., sodium fluoride, potassium fluoride, tin fluoride, disodium monofluorophosphate (Na₂PO₃F), dipotassium monofluorophosphate or the fluoride of an organic amino compound.

Antimicrobial active substances

Examples of suitable antimicrobial components are phenols, resorcinols, bisphenols, salicylanilides, salicylamides and their halogenated derivatives, halogenated carbanilides and p-hydroxybenzoates. Particularly suitable among the antimicrobial components are those which hinder the growth of plaque bacteria. For example, halogenated diphenyl ethers, such as 2,4-dichloro-2′-hydroxydiphenyl ether, 4,4′-dichloro-2′-hydroxydiphenyl ether, 2,4,4′-tribromo-2′-hydroxydiphenyl ether or 2,4,4′-trichloro-2′-hydroxydiphenyl ether (triclosan), are suitable as antimicrobial active substances. Apart from bromochlorophen, bisbiguanides, such as chlorohexidine and alexidine, phenylsalicylates and 5-amino-1,3-bis(2-ethylhexyl)hexahydro-5-methylpyrimidine (hexetidine), zinc and copper ions also act antimicrobially, synergistic effects, in particular in combination with hexetidine and triclosan, occurring. Quaternary ammonium compounds, such as, e.g., cetylpyridinium chloride, benzalkonium chloride, domiphen bromide and dequalinium chloride, can also be used. Octapinol, octenidine and sanguinarine have also proven to be antimicrobially effective. The antimicrobial active substances are preferably used in amounts of 0.01-1% by weight in the preparations according to the invention. Particular preference is given to the use of Irgacare® MP (triclosan) in an amount of 0.01-0.3% by weight.

Scale inhibitors

Scale consists of mineral deposits which are very similar to the natural dental enamel. In order to inhibit scale formation, substances are added to the dental cleaning preparations according to the invention which specifically interfere in the seed crystal formation and prevent the further growth of seeds already present. In this connection, they are, for example, condensed phosphates preferably selected from the group of the tripolyphosphates, the pyrophosphates, the trimetaphosphates or their mixtures. They are used in the form of their alkali metal or ammonium salts, preferably in the form of their sodium or potassium salts. Aqueous solutions of these phosphates typically have an alkaline reaction, so that the pH value of the dental care preparations according to the invention is adjusted, if appropriate by addition of acid, to values of 4-7.5. In this connection, use may be made as acids of, e.g., citric acid, phosphoric acid or acid salts, e.g. NaH₂PO₄. The desired pH value of the dental care preparation can, however, also be adjusted by addition of acid salts of the condensed phosphates, thus, e.g., K₂H₂P₂O₇.

Mixtures of different condensed phosphates and/or hydrated salts of the condensed phosphates can also be used according to the invention. Scale inhibitors are usually used in amounts of 0.1-5% by weight, preferably 0.1-3% by weight and in particular 0.1-2% by weight in the preparations according to the invention.

Additional suitable scale inhibitors are organophosphonates, such as 1-azacycloheptane-2,2-diphosphonate (Na salts) or 1-hydroxyethane-1,1-diphosphonate (Na salts), and zinc citrate.

Remineralization active substances

The preparations according to the invention preferably also comprise substances which promote remineralization of the dental enamel and make it possible to heal dental lesions. These are usually present in a total amount of 0.1-10% by weight, preferably 0.1-5% by weight and especially 0.1-3% by weight. These include, e.g., fluorides, phosphate salts of calcium, such as, e.g., calcium glycerol phosphates, calcium hydrogenphosphate, hydroxylapatite, fluoroapatite, F-doped hydroxylapatite, dicalcium phosphate dihydrate and calcium fluoride. However, magnesium salts, such as, e.g., magnesium sulfate, magnesium fluoride or magnesium monofluorophosphate, also act in a remineralizing fashion.

Flavoring agents

The preparations according to the invention preferably comprise flavoring agents including, e.g., sweeteners and/or aromatic oils. Examples of suitable sweeteners are saccharinates (in particular sodium saccharinate), cyclamates (in particular sodium cyclamate), and sucrose, lactose, maltose or fructose. All natural and synthetic flavorings conventional for oral and dental care preparations are possible as aromatic oils. Natural flavorings can be used both in the form of the essential oils isolated from the drugs (mixture) and in the form of the individual components isolated therefrom, Preferably, at least one aromatic oil from the group consisting of peppermint oil, spearmint oil, anise oil, star anise oil, caraway oil, eucalyptus oil, fennel oil, cinnamon oil, clove oil, geranium oil, sage oil, pimento oil, thyme oil, marjoram oil, basil oil, citrus oil, wintergreen oil or one/several components of these oils isolated therefrom or synthetically produced should be present. The most important components of the abovementioned oils are, e.g., menthol, carvone, anethole, cineole, eugenol, cinnamaldehyde, caryophyllene, geraniol, citronellol, linalool, salvene, thymol, terpinene, terpinol, estragole and methyl salicylate. Additional suitable flavorings are, e.g., menthyl acetate, vanillin, ionones, linalyl acetate, rhodinol and piperitone.

Sweeteners

Examples of suitable sweeteners are saccharin sodium, sodium cyclamate, acesulfame-K, aspartame, lactose, maltose and fructose, which are used in the preparations according to the invention in an amount of 0.05 to 0.5% by weight, preferably in an amount of approximately 0.2% by weight.

The dental bleaching preparation according to the invention can preferably comprise a series of additional components. These include, inter alia:
vitamins, e.g. retinol, biotin, tocopherol and their derivatives (e.g. esters, salts);
pigments, e.g. titanium dioxide or zinc oxide;
colored pigment particles, for example colored silica particles, such as those commercially available, e.g., under the sales designation Sorbosil®BFG 51, BFG 52 and BFG 53 or Sorbisol®2352. Mixtures of differently colored pigment particles can also be used. Such, e.g. rich orange, red or blue, colored gel silica particles can be present in amounts of 0.1-1.0% by weight in the preparations according to the invention;
dyes;
pH modifiers and buffering agents, e.g. sodium citrate, sodium bicarbonate or potassium and sodium phosphates, phosphoric acid and NaOH;
sodium benzoate;
wound-healing and antiinflammatory agents, such as, e.g., allantoin, urea, panthenol, azulene or camomile extract, or acetylsalicylic acid derivatives,
zinc sulfate.

The following examples should more fully describe the subject matter of the invention (the amounts indicated in the description and in the examples refer, unless otherwise specified, to % by weight, based on the total weight of the preparation).

### EXAMPLES

1) Water-rich dental bleaching gel with hydrogen peroxide

| Component | | % by weight |
|---|---|---|
| Glycerol (99.5%) | | 17.00 |
| 1,2-Propanediol | | 5.00 |
| Water, demineralized | | 46.81 |
| Phosphoric acid (85%) | | 0.15 |
| Disodium phosphate, anhydrous | | 1.20 |
| Sodium benzoate | | 0.04 |
| Saccharin sodium | | 0.20 |
| Sodium hydroxide, pearls | | 0.60 |
| Water, demineralized | | 4.00 |
| Azacycloheptanediphosphonic acid | | 0.50 |
| Polyethylene glycol (MW 400) | | 5.00 |
| Carbopol 974 PNF ® ¹ | | 1.50 |
| Hydrogen peroxide ® ² | | 18.00 |
| pH value: 6.9 | | |
| Water content: approximately 65% | | |
| Viscosity: | a) after 1 hour | 26 500 mPa · s |
| | b) after 3 hours | 27 500 mPa · s |
| | c) after 3 days | 33 500 mPa · s |

2) Water-poor dental bleaching gel with hydrogen peroxide

| Component | | % by weight |
|---|---|---|
| Glycerol (99.5%) | | 56.64 |
| Water, demineralized | | 2.00 |
| 1,2-Propanediol | | 5.00 |
| Phosphoric acid (85%) | | 0.10 |
| Disodium phosphate, anhydrous | | 1.50 |
| Trisodium phosphate, anhydrous | | 0.50 |
| Sodium benzoate | | 0.10 |
| Azacycloheptanediphosphonic acid | | 0.50 |
| Sodium hydroxide, pearls | | 0.16 |
| Water, demineralized | | 4.00 |
| Polyethylene glycol (MW 400) | | 10.00 |
| Carbopol 974 PNF ® ¹ | | 1.50 |
| Hydrogen peroxide ® ² | | 18.00 |
| pH value: 6.81 | | |
| Water content: approximately 20% | | |
| Viscosity: | a) after 1 hour | 16 625 mPa · s |
| | b) after 3 hours | 22 000 mPa · s |
| | c) after 3 days | 36 250 mPa · s |

3) Water-poor dental bleaching gel with hydrogen peroxide

| Component | % by weight |
|---|---|
| Glycerol (99.5%) | 58.64 |
| Water, demineralized | 2.00 |
| 1,2-Propanediol | 5.00 |
| Phosphoric acid (85%) | 0.10 |
| Sodium benzoate | 0.10 |
| Azacycloheptanediphosphonic acid | 0.50 |
| Sodium hydroxide, pearls | 0.16 |
| Water, demineralized | 4.00 |
| Polyethylene glycol (MW 400) | 10.00 |
| Carbopol 974 PNF ® ¹ | 1.50 |
| Hydrogen peroxide ® ² | 18.00 |
| pH value: 4.19 | |
| Water content: approximately 20% | |

4) Water-poor dental bleaching gel with carbamide peroxide

| Component | % by weight |
|---|---|
| Glycerol (99.5%) | 59.14 |
| Water, demineralized | 2.00 |
| 1,2-Propanediol | 5.00 |
| Phosphoric acid (85%) | 0.10 |
| Disodium phosphate, anhydrous | 1.50 |
| Trisodium phosphate, anhydrous | 0.50 |
| Sodium benzoate | 0.10 |
| Sodium hydroxide, pearls | 0.16 |
| Water, demineralized | 4.00 |
| Azacycloheptanediphosphonic acid | 0.50 |
| Polyethylene glycol (MW 400) | 10.00 |
| Carbopol 974 PNF ® ¹ | 1.00 |
| Carbamide peroxide ® ³ | 16.00 |
| pH value: 6.81 | |
| Water content: approximately 7% | |
| Viscosity: 31 days: 19 000 mPa · s | |

5) Water-poor dental bleaching gel with carbamide peroxide

| Component | % by weight |
|---|---|
| Glycerol (99.5%) | 58.9300 |
| Water, demineralized | 2.0000 |
| 1,2-Propanediol | 5.0000 |
| Phosphoric acid (85%) | 0.1000 |
| Disodium phosphate, anhydrous | 1.5000 |
| Trisodium phosphate, anhydrous | 0.5000 |
| Sodium benzoate | 0.1000 |
| Dragocolor fast green powder | 0.0026 |
| pigment | |
| Sodium hydroxide, pearls | 0.1600 |
| Water, demineralized | 4.0000 |
| Azacycloheptanediphosphonic acid | 0.5000 |
| Polyethylene glycol (MW 400) | 10.0000 |
| Carbopol 974 PNF ® ¹ | 1.0000 |
| Carbamide peroxide ® ³ | 15.2000 |
| Flavoring | 0.5000 |
| Tagat CH 60 ® ⁴ | 0.5000 |
| pH value: 6.70 | |
| Water content: approximately 7% | |

6) Water-rich dental bleaching gel with carbamide peroxide

| Component | % by weight |
|---|---|
| Glycerol (99.5%) | 19.00 |
| 1,2-Propanediol | 5.00 |
| Water, demineralized | 46.81 |
| Phosphoric acid (85%) | 0.15 |
| Disodium phosphate, anhydrous | 1.20 |
| Sodium benzoate | 0.04 |
| Saccharin sodium | 0.20 |
| Sodium hydroxide, pearls | 0.60 |
| Water, demineralized | 4.00 |
| Azacycloheptanediphosphonic acid | 0.50 |
| Polyethylene glycol (MW 400) | 5.00 |
| Carbopol 974 PNF ® ¹ | 1.50 |
| Carbamide peroxide ® ³ | 16.00 |
| pH value: 6.81 | |
| Water content: approximately 51% | |
| Viscosity: after 1 hour 7500 mPa · s | |

¹Polyacrylic acid, weakly crosslinked with allylpentaerythritol (Noveon Inc.)
²30% solution (Perhydrol ® ) (Merck KGaA)
³Product from Degussa
⁴Hydrogenated castor oil, ethoxylated with 60 mol of ethylene oxide (Goldschmidt)

Applicators

Since the abovementioned bleaching preparations, in contrast to other known systems, exhibit simply one component to be used, they are distinguished, in comparison with these, in particular by a particularly convenient handling during application. In addition, the composition (respectively the bleaching preparation) has a comparatively good storage stability. Because of the consequently relatively uncomplicated composition, several technically different kinds of applicators present themselves, which are to be preferred according to the properties of the respective composition, in particular depending on its viscosity. In this connection, it may be noted, at this point, that the viscosities given subsequently correspond to measured values recorded with a Brookfield RVF Helipath, the spindle 4/4 rpm being used up to 40 000 mPa·s and the spindle TE/4 rpm being used from 40 000 mPa·s.

It is also to be noted that, for entirely one and the same composition, several kinds of applicators can be used, the various applicators differing with regard to their operation. The manufacturer can consequently offer the user the possibility, according to preference, of choosing from several applicators. All applicators are distinguished in this connection by a more or less elastic applicator head with which the user can treat the surfaces of his teeth. In order to make possible a particularly specific application which accordingly is considerate of the surrounding gum, it is particularly advantageous if the applicator head exhibits an application area which is smaller than a normal tooth surface area. Advantageously, the width and the length of the application area are in each case less than 0.5 cm.

The kinds of applicators to be preferred, which are particularly advantageous for the application of the composition according to the invention to the teeth of the user, applicators being concerned which find use out office, thus outside the dental surgery, are described subsequently. The advantage of all applicators is that they require no additional means, such as impression molds or adhesive tapes, which remain in the mouth during the application. In this respect, it entails, on the one hand, any individual applicator but, on the other hand, the possibility of selecting from several applicators, which is highly convenient for the user.

Pen applicators

Mention may first be made of applicators which have approximately the shape of a writing pen and consequently are referred to subsequently as pen applicators. Such pen applicators have generally also already been known for a fairly long time in connection with the application of oily liquid and creamy cosmetics, for example from DE 2 260 724 A1 or EP 1 010 379 B1. They are suitable in particular for the application of the composition according to the invention to the teeth where the properties of the composition, depending on their formulation, lie in the range between a preferably somewhat oily-liquid (at 1000 mPa·s) and a pasty or jelly-like consistency (120 000 mPa·s). With the pen applicators, the composition can be applied within the entire viscosity range according to the invention, the use of the pen being preferred, because of the then simpler mechanistic construction, for the viscosity range of greater than 10 000 mPa·s. In addition, this kind of applicator is suitable for the pH value of the composition, which according to the invention lies in the range between 4 and 7.

The pen applicator has a reservoir for the composition and an application element connected to the reservoir via a feeder, the application element being formed, for example, into a little sponge or a plastic cylinder provided with channels and in particular being provided with a cover with which it acts upon the teeth. The application element and the cover form an applicator head. In addition, a piston is present which goes in a sliding fashion into the cylindrical reservoir and which can be slid by the user into the reservoir via an actuator, so that the composition is squeezed via the feeder into the application element and is available for use. In this connection, it may be advantageous to provide an arrangement with which the volume of the feeder can be somewhat reduced, in order to create a reservoir for the discharge of a single-use portion.

The particular advantage of the pen applicators is that their construction can be adapted with regard to the viscosity of the composition. Thus, for example, the diameter of the feeder can be correspondingly reduced with declining viscosity in order to prevent the composition from running out. In addition, the surface area and the permeability of the application element can be tuned to the viscosity.

In an advantageous embodiment, the pen applicator exhibits an arrangement whereby portioning of the exiting product is possible. This can be achieved, for example, via a catch arresting device while driving the piston. From the sound of the catch, the user notes that the piston has gone further by one unit and consequently a portion has been discharged. It is particularly advantageous if the reservoir of the applicator exhibits a volume of 4-5 ml, since the corresponding amount of composition makes possible a 14-day application.

Since conventional pen applicators can incorporate merely a volume of approximately 2 ml, it is advantageous, for a treatment cycle, to provide two applicators and to present these filled in a joint package. In this connection, the composition present in the one applicator can be provided for the morning application and the composition present in the other applicator can be provided for the evening application, the compositions being able to differ in their formulation.

Even more characteristics advantageous for the user can be achieved with the pen applicator. Thus, it is possible to intend the applicator for reuse. With regard to this, the composition, as with a fountain pen and an insulin pen, is filled into cartridges which can be inserted into the applicator. The cartridges can form a cylinder with a movable base which is acted upon by a stem and, like a piston, can slide in the cartridge case. The use of cartridges is advantageous for several reasons. Since a 14-day application and accordingly a volume of approximately 4-5 ml is required to achieve the desired bleaching effect, conventional pen applicators, as explained above, but can be filled with only approximately 2 ml, this problem can be solved with the use of two to three cartridges. In this connection, the use of the cartridges is sensible not only from economic considerations but also from ecological considerations. An applicator which can be filled with cartridges can also be used for an additional treatment cycle at a later date. An additional point, which the use of cartridges makes advantageous, is that these can be manufactured from glass, which in this respect contributes to increasing the shelf life as glass is considerably more resistant than plastic to the composition.

By and large, it is advantageous if, in the case of the use of cartridges in these or, however, in conventional applicators, a defined leakiness, in particular by a valve mechanism which can be penetrated in one direction, is provided, so that an excess pressure possibly building up by degassing can escape without problems.

In order to improve the hygienic conditions and to be able to adapt the application to the conditions in the mouth of the user, it is advantageous, in the pen applicator, to provide for a replaceable application element. Thus, in the application elements, the thickness of the lines or the angle of the application surface formed by the cover can vary in particular. Accordingly, it can be guaranteed that more inaccessible regions of the teeth, such as deep interdentia, can also be coated with the composition and that the surrounding gum is detrimentally affected as little as possible. On the whole, a product can be prepared with great convenience for the user with the use of the pen applicator.

Swab applicators

Applicators of this kind are comparable with the well-known cotton buds, the shaft of which, formed into a small tube, is filled with the composition, the liquid being brought, before application, from the shaft into the applicator head and the user coating the teeth with the impregnated applicator head. Applicators of this kind are in principle also known in connection with pharmaceutical and cosmetic preparations. These single-use applicators, formed into small pens, can be used in a different embodiment and, in their field of application, offer particular advantages in connection with the composition according to the invention.

First, it may be noted that the swab applicators to be described subsequently are preferably used for compositions, the viscosities of which lie in the lower range according to the invention. Thus, those swab applicators in which the composition, after the opening of the small tube, flows autonomously into the applicator head, in the range of the viscosity between 1000 mPa·s and 5000 mPa·s, in particular in the range between 1000 mPa·s and 2000 mPa·s, are particularly advantageous. On the other hand, in the range between 5000 mPa·s and 120 000 mPa·s, in particular between 5000 mPa·s and 20 000 mPa·s, use is advantageously made of those swab applicators in which the composition is “actively” displaced from the small tube into the applicator head by the user by means of a pen formed into a piston. Applicators of this kind, with their small tube generally manufactured from a chemically inert plastic, can be used without problems for the composition with a pH value in the range according to the invention between 4 and 7.

The advantage of the swab applicators generally lies first in their single-use character, which guarantees a maximum of hygiene to the user. In addition, the composition can, with applicators of this type, be preportioned in accordance with the single-use application, so that the user does not have to give any thought to possible under- or overcharging. An additional advantage of the swab applicators is the good handling, which guarantees a specific application of the composition to the tooth surface and accordingly the greatest possible protection of the gum. An additional advantage of applicators of this kind is that they, on the one hand, are satisfactorily sealable against the composition running out and diluting but, on the other hand, exhibit a degree of penetrability for possible degassing. In addition, it is advantageous for the applicators to be able to be marketed together in a common external packaging without packaging being required for each individual applicator. This guarantees, on the one hand, economical manufacture and, on the other hand, comfortable handling. The preparation of the applicators is comparatively simple and the filling can be metered in exactly.

In a particularly advantageous embodiment, the end of the small tube opening toward the applicator head is stoppered with a plug of highly viscous insoluble material, in particular made of silicone, which, when the applicator is used, opens a channel for the composition, from which the composition can flow into the applicator head. In this connection, the plug is held on the walls of the small tube or gets caught before the applicator head on rinsing. Such a plug improves the leaktightness toward seepage and evaporation of the composition.

As already explained, there are different embodiments of the swab applicators, to which, however, a slender small tube as container and an applicator head mounted thereon are common. The differences are in the way in which the applicator head is charged with the composition. Both types of swab applicators are described later in connection with the exemplary embodiments.

Rod applicator in the vial

An additional possibility for applying the composition is the use of a device which exhibits a rod applicator with applicator head positioned at the end of a small rod, which is dipped into a vial comprising the composition. In this connection, the rod applicator and the vial in this respect form a unit when the filled vial is stoppered with the rod applicator. In this situation, the applicator head is immersed in the composition. Similar applicators are also known in connection with cosmetic products, for example with nail varnish.

With these applicators, compositions can in principle be used over the entire range according to the invention of the viscosities, being suitable in particular for the oily liquid compositions with viscosities between 1000 mPa·s and 5000 mPa·s, in particular in the range between 1000 mPa·s and 2000 mPa·s. With jelly-like compositions in the range of the viscosities above 5000 mPa·s, in particular above 8000 mPa·s, it is advantageous to provide, inside the vial, means which strips from the applicator head, when the applicator rod is withdrawn, an excess amount formed of the composition. Thus, precise dosing can be guaranteed. With viscosities above 50 000 mPa·s, the rod applicator is only suitable up to a point. In addition, applicators of this kind can be used for the composition with a pH value in the range according to the invention between 4 and 7.

The particular advantage of these applicators is first that they can be manufactured with volumes of any size. It is thus possible without problems to fill such a vial with the amount of approximately 5 ml necessary for a 14-day application. In this connection, it is advantageous to provide the user with a considerably greater amount, for example twice the amount, since this method of application is not exactly economical to the users. Thus, the users are rather inclined to dip the applicator several times in the vial, even if a sufficient amount of the composition is actually yet present on the applicator head. The greater amount in the vial also makes possible repeated application on the one day.

An additional advantage of these applicators is their uncomplicated construction. Consequently, they can be manufactured and filled particularly economically in any form. In a particularly simple case, the applicator pen is fixed to the inner base of a screw cap which, to close the vial, is screwed onto its neck in a leaktight fashion. The vial with the screw cap also offers great leeway for the layout thereof, so that the product can be individualized particularly well with regard to its design. An additional advantage of the vial with a screw cap is the leaktightness, which results in a high storage stability. In addition, the comparatively large space above the liquid forms an extraction chamber for possible degassing components which then escape in a controlled fashion on unscrewing. Consequently, the vial with a screw cap, in which a defined leakiness can additionally be provided, for example in the lid or in the screw thread, is an ideal container for the composition according to the invention. It happens that the container can be manufactured not only in plastic but also in glass, which further increases the storage stability.

In addition, it is advantageous that the applicators, in particular the applicator heads, can be adjusted particularly well to the respective requirements. In this connection, the material of the applicator head can be selected in accordance with the viscosity in such a way that the correct amount of the composition remains attached. For this, the head can be manufactured from a more or less porous foamed material which absorbs the composition like a sponge. It has proved to be the case that the use of a small brush as applicator head is less suitable, since the act of coating on the teeth by the user cannot be controlled particularly well. The advantage of the applicator head cut out from such a material is also that its shape can have any form. Thus, broader or narrower stripping surfaces in any angle of attack or with concave curvature adapted to the tooth surface can be provided. With such applicator heads optimized with regard to their shape, problem areas in the set of teeth of the user can be satisfactorily reached.

If less advantageous, the use of the composition in connection with other applicators is naturally not excluded. Thus, at a particularly high viscosity, the creamy composition can be poured into a cylindrical mold and can be applied using a kind of lipstick with a central part which can be unscrewed. At a particularly high viscosity of the composition, it is also conceivable to provide pencils which can be sharpened, in the manner of kohl pencils, the “lead” of which is formed from the composition. In the case of low viscosities, it is also conceivable to fill the liquid composition under pressure into a small spray bottle or, without pressure, into a pump dispenser.

### DESCRIPTION OF THE DRAWINGS

Different embodiments of suitable applicators are more fully represented subsequently from FIGS. 1 to 5.

FIG. 1 shows a pen applicator,

FIG. 2 shows a pen applicator with cartridge,

FIG. 3 shows a swab applicator with movable piston in the cylinder,

FIG. 4 shows a swab applicator and

FIG. 5 shows a rod applicator with vial.

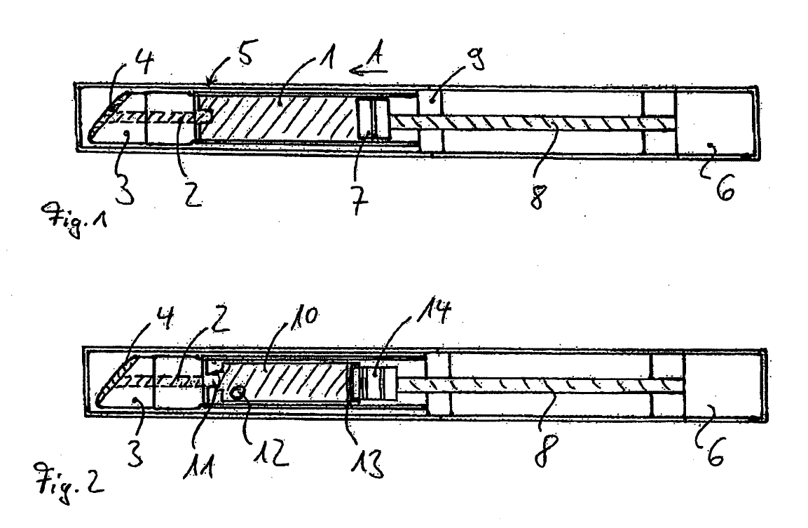

FIG. 1 shows a pen applicator which is approximately as large as a conventional writing pen and which exhibits a reservoir 1 for the storage of the composition according to the invention (hatched). The reservoir 1 is situated above a feeder 2 connected to an application element 3 which, on its face, exhibits a small sponge as cover 4. The application element 3 is, in the example represented, covered with a removable cap 5. As soon as this cap 5 is removed, the user can coat his teeth with the cover 4. However, beforehand, he has to displace a piston 7, which goes in a sliding fashion into the cylindrical reservoir 1, in the direction of the arrow A by rotating a button 6 attached to the end of the pen and consequently has to squeeze the composition from the reservoir 1 into the application element 3. In this connection, the piston 7 is driven via a stem 8 which goes into a nut 9. The button 6, the stem 8, the piston 7 and the nut 9 represent an actuator. In this embodiment, the pen applicator has, when the piston is driven, a catch arresting device (not represented) with which portioning of the exiting product is possible. The user notes, from the snapping noise, that the piston has each time gone further by one unit and in the course of this has discharged one portion.

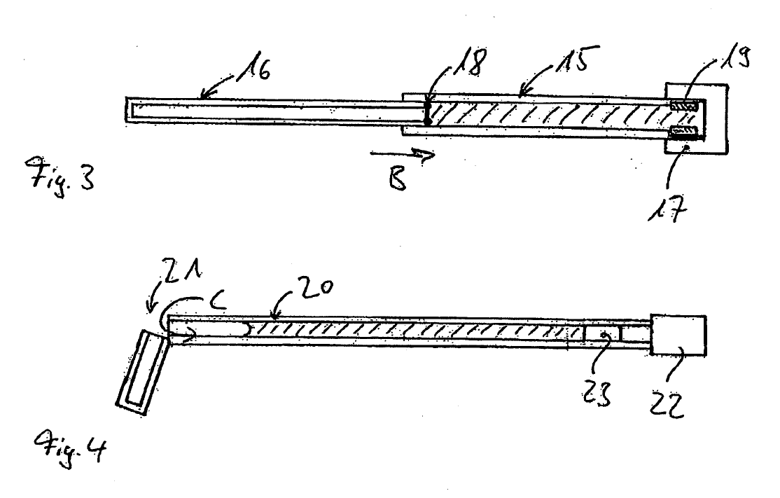

In principle, the same pen applicator is shown in FIG. 2 as in FIG. 1 yet, in this case, the composition is not stored in a static reservoir but in an exchangeable cartridge 10. To change the cartridge 10, the pen is unscrewed like a fountain pen, the piston 7 first having been retracted into its starting position. The cartridge 10 is then positioned on a nozzle 11 which, during the positioning, opens a closing device of the cartridge, the closing device in this case being formed by a valve ball 12 which is forced back into the cartridge 10. The cartridge forms a cylinder with a movable base 13 which is acted upon by a punch 14 located on the head of the stem 8 and thus, like a piston, can be pushed into the cartridge case.

A swab applicator is shown in FIG. 3, which applicator, in the pushed-together condition, is approximately the size of a well-known cotton bud. The applicator first exhibits a shaft 15 formed into a small tube which is filled with the composition (hatched). The shaft 15 forms a cylinder in which a pen 16 can move as a piston in the direction of the arrow B. In the course of this, it slides into the cylindrical shaft with a leaktight element 18 situated on the head of the pen 16. By pressing on the pen 16, the latter is displaced and drives the liquid located in the shaft 15 into an applicator head 17 which is manufactured from a porous material, for example a foamed material, and is saturated by the liquid composition. The user coats his teeth with the applicator head 17 thus impregnated.

In this example, a silicone plug 19 is introduced into the shaft 15, which plug, on pressing on the shaft, is penetrated by the composition before the latter saturates the applicator head 17. This single-use applicator is, before use, stored in the condition represented in a joint packaging together with the additional single-use applicators necessary for the “therapy”.

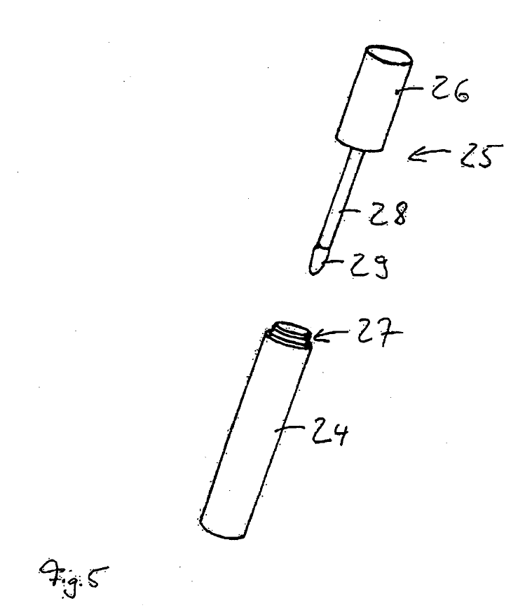

FIG. 4 shows a swab applicator which exhibits a small tube 20 made of plastic which is filled with composition. In the initial condition, the small tube 20 is closed at the rear end 21. An applicator head 22 made of porous material, which is impregnated before use with the composition, is, for its part, attached to the front opening end of the small tube 20. A plug 23 made of silicone, which prevents evaporation of the composition, is also, in this case, positioned in the small tube in front of the head. In FIG. 4, the swab applicator is represented shortly before it is used. In this situation, the rear end 21 is broken at a predetermined breaking point so that air can force its way in (arrow C) and the composition can flow into the applicator head 22. The composition first penetrates the plug 23 and then saturates the applicator head 22 from inside. In this condition, the swab applicator is ready for use.

FIG. 5 represents an applicator which exhibits a vial 24 filled with the composition and a rod applicator 25 which can be inserted into the vial. The rod applicator 25 has a closure part 26 which is attached to the vial and is closed in a leaktight way by means of a screw thread 27. A small rod 28 is positioned in the base of the closure part 26 and an applicator head 29 is fixed to the end of this small rod. If the vial 24 is closed, the small rod 28 with the applicator head 29 dips into the composition so that the applicator head 29 is saturated, The user coats his teeth with the saturated applicator head 29. Stripping means (not represented), which strip off possible excess material from the applicator head 29, may be provided inside the vial.

1. A preparation for bleaching teeth, comprising
0.3 to 30% by weight of a bleaching component selected from hydrogen peroxide or carbamide peroxide,
4 to 80% by weight of water,
0.05 to 1.00% by weight of a complexing agent selected from the group of the biphosphonates, and
0.1 to 3.0% by weight of a thickener,
which exhibits a pH value in the range from 4 to 7 and a viscosity in the range from 1000 to 120 000 mPa·s (measured with a Brookfield RVF; up to 40 000 mPa·s spindle 4/4 rpm; from 40 000 mPa·s spindle TE/4 rpm; Helipath).

2.-29. (canceled)
